(19) United States
(12) Patent Application Publication
(10) Pub. No.: US 2019/0234938 A1
(43) Pub. Date: Aug. 1, 2019

(54) COMPOSITION FOR EVALUATING IMMUNE RESPONSE INDUCING ABILITY OF DRUG

(71) Applicants: YOUNG-SOO SHIN, Cerritos, CA (US); BONG-HA SHIN, Cerritos, CA (US); KYOO-HO SHIN, Cerritos, CA (US)

(72) Inventors: YOUNG-SOO SHIN, Cerritos, CA (US); BONG-HA SHIN, Cerritos, CA (US); KYOO-HO SHIN, Cerritos, CA (US)

(21) Appl. No.: 16/339,350

(22) PCT Filed: Feb. 21, 2018
(86) PCT No.: PCT/KR2018/002141

§ 371 Date: Apr. 3, 2019

(30) Foreign Application Priority Data
Feb. 21, 2017 (KR) 10-20170023123

(51) Int. Cl. G01N 33/50

(52) U.S. Cl. CPC G01N 33/5047; G01N 2800/52

(57) ABSTRACT

The present invention relates to a composition for evaluating the immune response of blood of a drug and a subject. The present invention also relates to a method for evaluating induction of an immune response of a drug using the composition.

### BACKGROUND OF THE INVENTION

### Field of the Invention

The present invention relates to a composition for evaluating immune response inducing ability of drug and a method for evaluating the ability of a drug to induce an immune response using the composition.

### Description of the Prior Art

An immune response is a vital reaction that defends the body from external infectious agents such as germs; the cells involved in this immune response are called immune cells. Immune cells include macrophages, B lymphocytes, T lymphocytes, helper-T lymphocytes, inhibitory T lymphocytes, natural killer cells (NK cells), NKT, and DC. After macrophages or cancer cells have been fed into macrophages, there are antigen presenting cells that present antigens on the surface of microorganisms or cancer cells. B lymphocytes recognize the presented antigen or microorganism and produce an antibody against them. Cytotoxic T lymphocytes directly destroy cells with external antigens; helper T lymphocytes regulate these immune responses.

All disease conditions (infection, cancer, etc.) activate our body's immune cells. However, the degree of activation and the pattern of the immune cells depend on the disease. For example, a rapid or overactive immune response (acute infection) could cause a sudden fever; in severe cases sepsis or the like can threaten the life of the patient. In addition, the chronic activation of immune cells induced by cancer and the like induces a basement of normal immune function.

Activation of the immune response begins with all immune cells producing their own energy. The mitochondria in each immune cell carry an immune response through the oxygen consumption around the most dust. That is, when the immune cells in the patient's blood are converted, the immune cells use oxygen in the blood (Karhausen et al., The Journal of Clinical Investigation, Vol.114, No. 8, October 2004, Epithelial hypoxia-inducible factor-1 is protective in murine experimental colitis). Therefore, the underground concentration of oxygen around the immune cells means the activation of immune cells.

Humankind is treating patients through numerous medications. The medical staff administers various drugs for treatment, but the drug ultimately treats the patient; how the drug affects restored immune cells cannot be known before administration. The degree to which a patient responds to a drug depends on the individual; this is because there is no method that can be preliminarily confirmed before drug administration. The medical staff judges whether or not the immune cell is activated according to the increase or decrease of the immune cell count; there is only universal knowledge that immunosuppressants will prevent the activation or proliferation of immune cells. Therefore, in actual clinical practice, the prescribed drug has only been used by adding or subtracting dosage according to sex, age, and weight.

This leads to the long-term use of unnecessary drugs that do not work well with patients, so that not only is the cost wasted, but it also causes drug resistance and drug side effects to the patient.

Therefore, it has been necessary to precisely know the specificity and sensitivity of the individual immune cells, especially the drug, before the administration of the drug. However, the immune cell testing that is currently being used is for the analysis of the number of immune cells in blood taken from patients, for the analysis of inhibitory substances or active substances of immune cells, or to observe the killing ability of specific immune cells by isolating specific immune cells and culturing them with other substances or cells, especially cancer cells. This is a way of indirectly and artificially estimating the function of immune cells and not a direct method viewing immune cell reaction in the blood. Moreover, the specificity and sensitivity of the drug are difficult to deduce precisely with the above methods.

Accordingly, the inventors of the present invention have been studying a method for confirming the immune response induction of immune cells of drugs, and in so doing have confirmed that the use of the composition of the present invention was able to accurately evaluate the immune response induction ability of the drug in the blood, thus completing the present invention.

### BRIEF SUMMARY OF THE INVENTION

### Problem that the Invention is to Solve

It is an object of the present invention to provide a composition, that is, a reagent, which is capable of evaluating the ability of a drug to induce an immune response in blood.

It is a further object of the present invention to provide a method for evaluating the ability of a drug to induce an immune response in blood.

### Means for Solving the Problems

In order to accomplish the above objects, the present invention provides a composition for evaluating the immune response of blood to a drug and a subject, comprising a vitamin B group, vitamin D and PBS.

Further, the present invention provides a method for evaluating the immune response induction ability of the drug comprising a step of mixing the composition for assessing the immune response of the present invention, and a step of confirming the immune response to the mixture.

### Effects of the Invention

The composition and the evaluation method of the present invention allow the immune response of an immune cell to a patient's individual drug to be known quickly and accurately before administration of the drug to the patient, thereby making it possible to prescribe safe and effective personalized drugs (antibiotics, anticancer drugs, etc.).

In addition, the use of the composition of the present invention can reveal changes in the individual immune response over time for certain drugs. Thus, by measuring the immune response periodically (for example, one or two times per month) information on how the individual immune response changes with the treatment period can be obtained. Therefore the present invention enables medical personnel to perform follow-up observations of the persistent immune system changes of the patient to prescribe safer and more effective treatment.

The composition and method of the present invention can also be used in drug development. That is, by preliminarily evaluating the ability of the candidate drug to induce an immune response by using the results of the reaction between the blood and the candidate drug, it becomes possible to prevent side effects that may occur with persons taking the drug, thereby making it possible to estimate the drug efficacy in advance. This enables the development of more effective drugs in a rapid manner.

### BRIEF DESCRIPTION OF THE DRAWINGS

FIG. 1 shows a plate for a drug reaction.

FIG. 2 shows a device for measuring PO₂ for the measurement of cellular monolayer PO₂ of the cell itself.

FIG. 3 is a view of injecting a mixture of a reagent and blood into a drug reaction plate.

FIG. 4 shows the oxygen consumption concentration when the blood of the patient A and the first reagent are mixed with the drug (A: azithromycin, C: ceftriaxone, D: doxycycline).

FIG. 5 shows the oxygen consumption concentration when the blood of patient A and RMPI 1640 medium are mixed with drug (A: azithromycin, C: ceftriaxone, D: doxycycline).

FIG. 6 shows the oxygen consumption concentration when the blood of patient B and the first reagent are mixed with the drug (A: azithromycin, C: ceftriaxone, D: doxycycline).

FIG. 7 shows the oxygen consumption concentration when the blood of patient B and RMPI 1640 medium are mixed with the drug (A: azithromycin, C: ceftriaxone, D: doxycycline).

FIG. 8 shows the oxygen consumption concentration when the blood of patient C and the first reagent are mixed with the drug (A: azithromycin, C: ceftriaxone, D: doxycycline).

FIG. 9 shows the oxygen consumption concentration when the blood of patient C and RMPI 1640 medium are mixed with the drug (A: azithromycin, C: ceftriaxone, D: doxycycline).

FIG. 10 shows the oxygen consumption concentration when the blood of patient D and the first reagent are mixed with the drug (D: dasatinib, N: nilotinib, I: imatinib).

FIG. 11 shows the oxygen consumption concentration when the blood of patient D and RMPI 1640 medium are mixed with the drug (D: dasatinib, N: nilotinib, I: imatinib).

FIG. 12 shows the oxygen consumption concentration when the blood of patient E and the first reagent are mixed with the drug (D: dasatinib, N: nilotinib, I: imatinib).

FIG. 13 shows the oxygen consumption concentration when the blood of patient E and RMPI 1640 medium are mixed with the drug (D: dasatinib, N: nilotinib, I: imatinib).

FIG. 14 shows the oxygen consumption concentration when the blood of patient F and the first reagent are mixed with the drug (D: dasatinib, N: nilotinib, I: imatinib).

FIG. 15 shows the oxygen consumption concentration when the blood of patient F and RMPI 1640 medium are mixed with the drug (D: dasatinib, N: nilotinib, I: imatinib).

FIG. 16 shows the oxygen consumption concentration when the blood of patient G and the first reagent are mixed with the drug (D: dasatinib, N: nilotinib, I: imatinib).

FIG. 17 shows the oxygen consumption concentration when the blood of patient G and RMPI 1640 medium are mixed with the drug (D: dasatinib, N: nilotinib, I: imatinib).

FIG. 18 shows the oxygen consumption concentration when the blood of patient H and the first reagent are mixed with the drug (A: Aspirin, C: corticosteroid, P: corticosteroidPenicillin).

FIG. 19 shows the oxygen consumption concentration when the blood of patient I and the first reagent are mixed with the drug (A: Aspirin, C: corticosteroid, P: corticosteroidPenicillin).

FIG. 20 shows the oxygen consumption concentration when the blood of patient J and the first reagent are mixed with the drug (A: Aspirin, C: corticosteroid, P: corticosteroidPenicillin).

### DETAILED DESCRIPTION

### Best Mode for Carrying Out the Invention

The present invention relates to a composition for evaluating the immune response of blood of a drug and a subject, comprising a vitamin B group, vitamin D and PBS.

Further, the present invention relates to a method for evaluating the immune response induction ability of the drug comprising a step of mixing a composition for evaluating an immune response, the blood of a subject and a drug, and a step of confirming the immune response to the mixture.

The present invention is described in detail below

The composition for assessing immune response of the present invention

The present invention relates to a composition for evaluating the immune response of blood of a drug and a subject, comprising a vitamin B group, vitamin D and PBS. The subject means a patient having a specific disease. By preparing a mixture by mixing the composition for assessing an immune response, the blood and the drug and measuring the degree of oxygen consumption in the mixture, the composition of the present invention is used to evaluate the immune response induction ability of the drug. At this time, the composition of the present invention preferably comprises vitamin B group, vitamin D and PBS in a volume ratio of 10:0.5 to 3:800 to 1300.

The composition for assessing an immune response of the present invention may further comprise at least one selected from the group consisting of anti-blood coagulants, plasma proteins, iron-binding plasma proteins, calcium, selenium, thrombin inhibitors and pyruvate.

The anti-blood coagulant may be aprotinin. The plasma protein may be fetuin. The iron-binding plasma protein may be a transferrin. In one example, the composition for evaluating an immune response of the present invention may further comprise at least one selected from the group consisting of aprotinin, fetuin, transferrin, calcium, selenium, thrombin inhibitor and pyruvate. At this time, the composition of the present invention preferably comprises vitamin B group, aprotinin, fetuin, transferrin, calcium, selenium, thrombin inhibitors and pyruvate in volume ratios of 10:0.05 to 0.3:0.07 to 0.4:0.5 to 3:0.05 to 0.3:0.05to 0.3:0.5 to 3:0.07 to 0.4. For example, the composition of the present invention may further comprise a vitamin B group: aprotinin in a volume ratio of 10:0.05 to 0.3. In addition, the composition of the present invention may comprise the vitamin B group: fetuin in a volume ratio of 10:0.07 to 0.4. In addition, the composition of the present invention may comprise the vitamin B group: transferrin in a volume ratio of 10:0.5 to 3. Further, the composition of the present invention may comprise vitamin B group: calcium in a volume ratio of 10:0.05 to 0.3. Moreover, the composition of the present invention may comprise the vitamin B group: selenium in a volume ratio of 10:0.05 to 0.3. Additionally, the composition of the present invention may comprise the vitamin B group: thrombin inhibitor in a volume ratio of 10:0.5-3. Furthermore, the composition of the present invention may comprise the vitamin B group: pyruvate in a volume ratio of 10:0.07 to 0.4.

The composition for evaluating an immune response of the present invention may additionally comprise one or more selected from the group consisting of the mixture of vitamins A, C, E and K, heparin, deldeparin sodium, argatroban, bivalirudin, Lepirudin, serum albumin and immunoglobulin. At this time, the composition of the present invention may comprise a vitamin B group, a mixture of vitamins A, C, E and K, heparin, daldeparin sodium, argatroban, bivalirudin, Lepirudin, serum albumin and immunoglobulin in a volume ratio of 10:2 to 7:0.05 to 0.3:0.05 to 0.3:0.05 to 0.3:0.05 to 0.3:0.05 to 0.5:0.07 to 0.8:0.07 to 0.8.

The vitamin B group of the present invention preferably comprises two or more selected from the group consisting of vitamin B1, vitamin B2, vitamin B3, vitamin B5, vitamin B7, vitamin B9 and vitamin B12.

Use of the composition for evaluating an immune response of the present invention

Immune cells require oxygen for activation, and oxygen consumption shows the activation of immune cells.

The composition of the present invention measures the consumption of oxygen in the blood in vitro and quantifies it, so that it is possible to know the degree and condition of immune reaction in blood during drug treatment. Therefore, the composition for evaluating an immune response of the present invention enables evaluation of the ability of a drug to induce an immune response in the blood upon treatment of the drug in blood.

By assessing the ability of the drug to elicit an immune response in the subject, that is, the blood of a patient with a particular disease, it can be predicted in advance whether or not it will be effective to prescribe the drug to the subject.

For example, it is desirable to administer antibiotics that cause a strong immune response in patients with Chlamydia infection. It is also desirable to administer an anti-cancer agent that causes a strong immune response in cancer patients. Particularly, in the case of cancer, a cocktail therapy is generally used in which various kinds of cancer drugs are mixed according to the kind of cancer; some combinations of drugs have no effect on the patient, but only cause severe side effects. Therefore, it is effective to exclude the drugs with insufficient anti-cancer effects that only cause side effects, and to select and administer an anticancer combination that induces a strong immune response.

On the other hand, patients with acute rheumatic fever (ARF) and rheumatic heart disease (RHD) are preferably given antibiotics that cause a weak immune response. The control of ARF is performed by a method of reducing inflammation with anti-inflammatory drugs such as aspirin or corticosteroids; patients who have ARF once will receive a one-time persistent antibiotic for 5 years.

The composition and the evaluation method of the present invention allow the immune response of an immune cell to a patient's individual drug to be known quickly and accurately before administration of the drug to the patient, making it possible to prescribe safe and effective personalized drugs (antibiotics, anticancer drugs, etc.). Further, the present invention enables a medical staff to keep track of a patient's continuous immune system changes to enable the prescription of safer and more effective treatment. The composition and method of the present invention can also be used in drug development. That is, by preliminarily evaluating the ability of the candidate drug to induce an immune response by using the results of the reaction between the blood and the candidate drug, side effects that may occur to drug users may be prevented in advance, and the drug efficacy can be estimated in advance. This allows the rapid development of more effective drugs.

By evaluating the immune response-inducing ability of a drug to be evaluated using the composition for evaluating immune response of the present invention, a drug having the desired immune response inducing ability, an optimal drug combination or an optimal dose can be found. This allows the medical staff to identify, prescribe and administer a customized drug for each patient before administering the drug to the patient.

In addition, in the stage of drug development, the composition and method of the present invention can be used to selectively screen medications having desired the immune response inducing ability in many patients.

Furthermore, by evaluating the immune response-inducing ability of a drug to be evaluated using the composition of the present invention, it is possible to pre-screen for drugs that are not effective with the patient; this has the effect of eliminating drugs that cause side effects and have no pharmacological effects, especially medications with side effects. In addition, the composition and method of the present invention can be used to accurately select drugs that protect patients from trouble with the immune system and that effectively aid immune function.

Method of Evaluating Immune Response Induction Ability of Drug

The present invention relates to a method of evaluating the immune response induction ability of a drug comprising a step of mixing a composition for evaluating an immune response with the blood and the drug of a subject, and a step of confirming the immune response in the mixture.

The blood is blood obtained by collecting blood from a subject. Therefore, the method of the present invention is carried out in vitro. The evaluation of the ability of the drug of the present invention to induce an immune response is performed by evaluating the degree to which the drug induces an immune response in the mixture; in particular, the degree to which the drug induces an immune response in the mixture is evaluated by measuring the degree of oxygen consumption in the mixture.

By evaluating the drug's ability to induce an immune response, the present invention enables the selection of drugs suitable for the subject by screening them. That is, the present invention can be a method for screening drugs suitable for treating a subject's disease by evaluating the ability of the drug to elicit an immune response comprising a step of mixing a composition for evaluating an immune response with the blood and a drug of a subject, and a step of confirming the immune response to the mixture.

Form for Embodying the Invention

<Materials and Method>

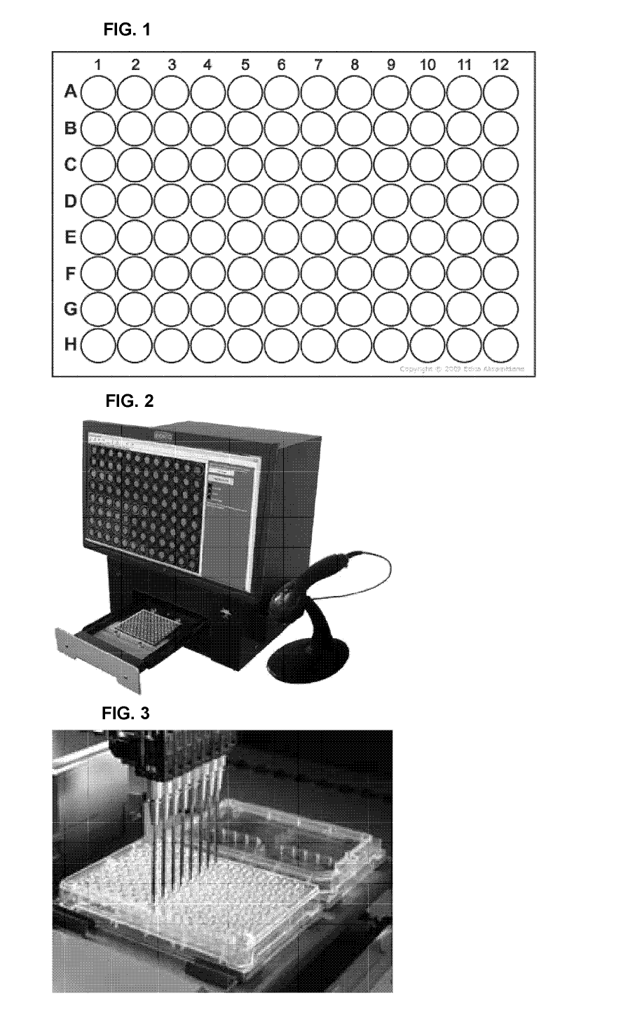

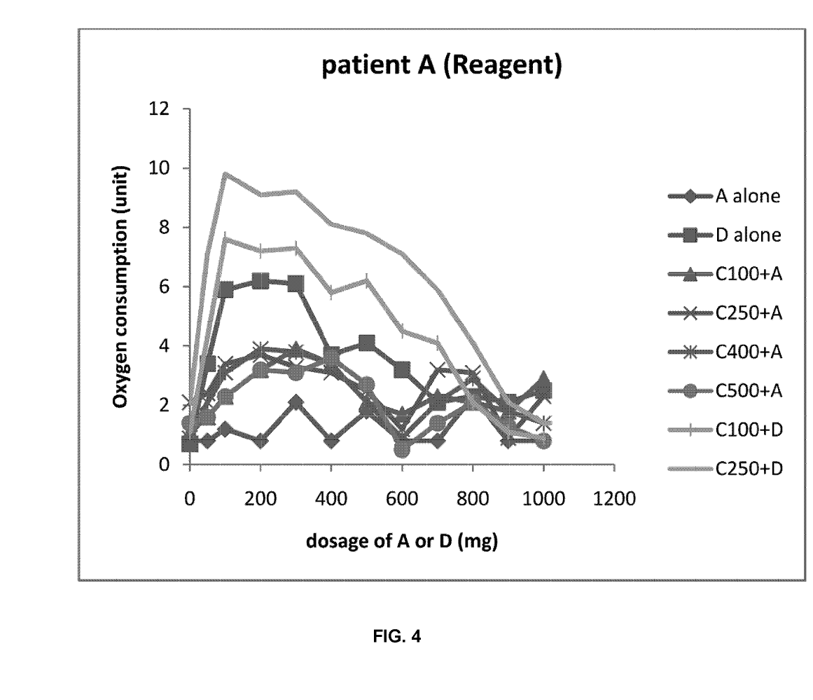

The drug reaction plate uses a plate having the structure shown in FIG. 1. In this case, drugs were administered differently according to the concentration of drug in the directions 1-12; in the A-H direction, the drug was administered according to the type or combination of drugs (anticancer drugs, antibiotics, etc.).

To measure the cellular monolayer PO₂ of the cell itself, a PO₂ measuring device was used (FIG. 2). For the vitamin B group, B2 (riboflavin), B3 (mixed with niacin, nicotinic acid and nicotinamide riboside in the same volume ratio) B6 (mixed pyridoxine, pyridoxal and pyridoxamine in the same volume ratio) and B12 (mixed with cyanocobalamin and methylcobalamin in the same volume ratio) were used in a volume ratio of 1:1:3:3 (that is, B2: B3: B6: B12 in a volume ratio of 1:1:3:3).

Vitamin A, C, E and K mixtures were also prepared by mixing vitamin A, C (ascorbic acid), E (tocopherol) and K in the same volume ratio.

At this time, as vitamin A, beta-carotene and gamma-carotene were used by mixing them at the same volume ratio, and as vitamin K, vitamin K1 and vitamin K2 were used by mixing them at the same volume ratio.

After blood was collected from the patient, 200 μl of blood was mixed with 100 μl of reagent (FIG. 3), the mixture was injected into the drug reaction plate and the drug was injected to cause the reaction of the blood, reagent and drug. This was stored at room temperature (22 to 24° C.) for about 1 hour to measure the SpO₂ concentration after the drug reaction.

As a control, RPMI 1640 medium, a commercially available synthetic culture medium, was used; 200 μl of blood and the drug were injected into the RPMI 1640 medium and SpO₂ concentration was measured after 1 hour.

### PREPARATION EXAMPLE 1

Vitamin B group, aprotinin, Fetuin, Transferrin, vitamin D, phosphate-buffered saline (PBS), calcium, selenium, thrombin inhibitor and pyruvate were mixed in a volume ratio of 10:0.1:0.2:1:1:985:0.1:0.1:1:0.2 to prepare a first reagent.

### PREPARATION EXAMPLE 2

Vitamin B group, aprotinin, Fetuin, Transferrin, vitamin D, phosphate-buffered saline (PBS), calcium, selenium, thrombin inhibitor, pyruvate, heparin, Delteparin sodium, Agatroban, Bivalirudin, Lepirudin, Serum albumin, Immunoglobulin, Vitamins A, C, E and K mixture were mixed in a volume ratio of 10:0.1:0.2:1:1:985: 0.1:0.1:1:0.2:0.1:0.1:0.1:0.1:0.1:0.2:0.2:4 to prepare a second reagent.

### EXPERIMENTAL EXAMPLE 1

### Testing of Patient Administered Antibiotics

Patient A with Chlamydia infection was treated with ceftriaxone (250 mg IM) and azithromycin (1000 mg orally), but the patient did not have improved symptoms. The blood of the infected patient A was collected in cooperation with the medical staff and the patient, and this was mixed with the reagent 1 of Preparation Example 1. Ceftriaxone, doxycycline and azithromycin were administered alone or in combination, and the SpO₂ concentration was measured after induction of the reaction for 1 hour.

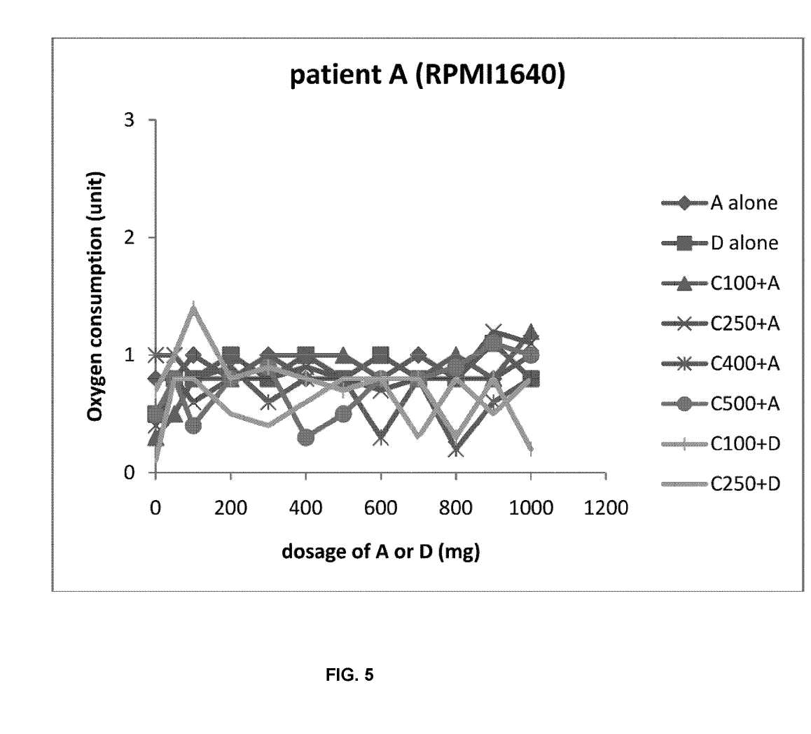

On the other hand, no significant difference was observed in the O₂ consumption concentration depending on the drug in the control group (FIG. 5).

Therefore, medical staff administered doxycycline instead of azithromycin to patient A (in other words, a combination of ceftriaxone and doxycycline); after two weeks of treatment, patient A recovered (A: azithromycin, C: ceftriaxone, D: doxycycline).

### EXPERIMENTAL EXAMPLE 2

### Testing of Patient Administered Antibiotics

Patient B with Chlamydia infection was treated with ceftriaxone (250 mg IM) and azithromycin (1000 mg orally), but the patient's symptoms were not improved. The blood of the infected patient B was collected in cooperation with the medical staff and the patient, and this was mixed with the reagent 1 of production example 1. Ceftriaxone, doxycycline and azithromycin were administered alone or in combination, and the SpO₂ concentration was measured after induction of the reaction for 1 hour.

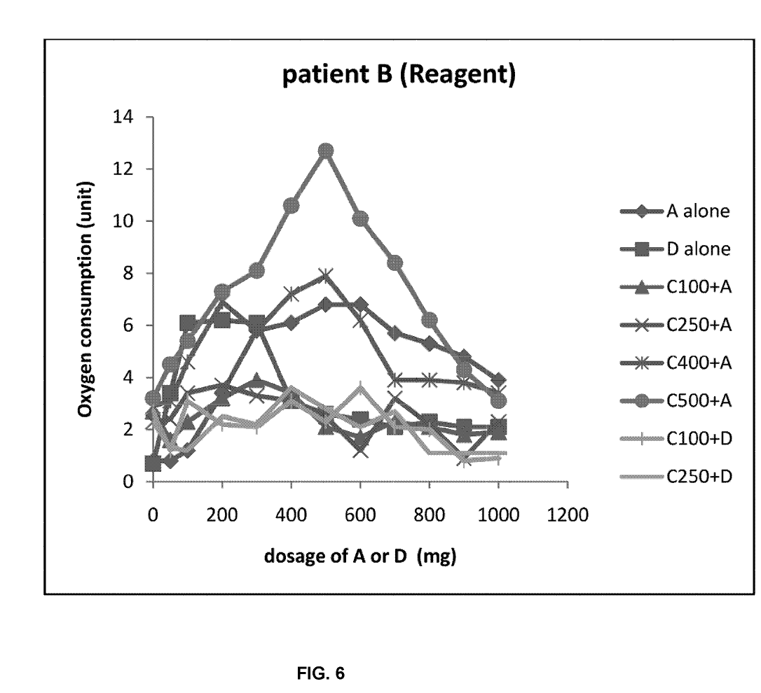

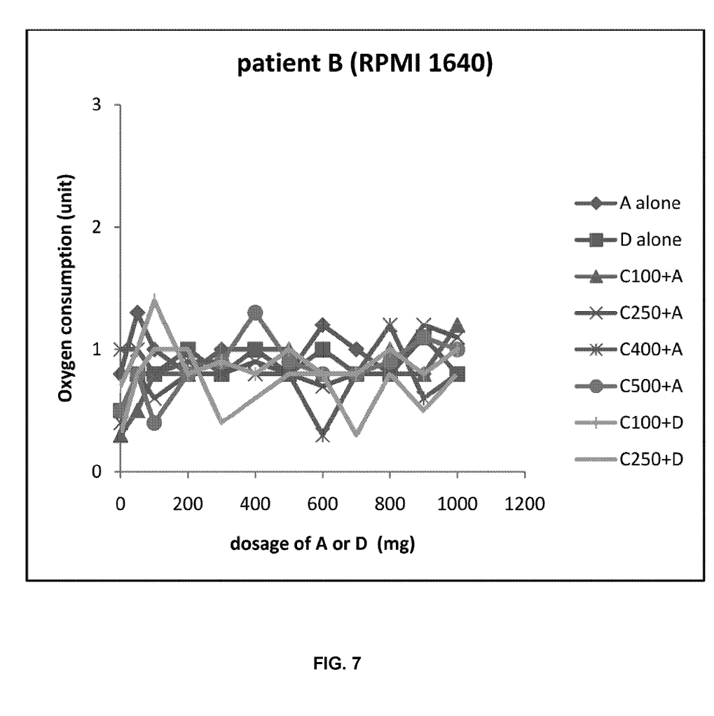

As a result, it was confirmed that, among the experimental groups using the first reagent, the consumption concentration of O₂ in the group treated with ceftriaxone 500 mg and azithromycin 500 mg was significantly higher (FIG. 6). On the other hand, no significant difference was observed in the O₂ consumption concentration depending on the drug in the control group (FIG. 7). Therefore, the medical staff increased the dose of ceftriaxone and decreased the dose of azithromycin to patient B; after 2 weeks of treatment, patient B recovered (A: azithromycin, C: ceftriaxone, D: doxycycline).

### EXPERIMENTAL EXAMPLE 3

### Testing of Patient Administered Antibiotics

Patient C with Chlamydia infection was treated with ceftriaxone (250 mg IM) and azithromycin (1000 mg orally), but the patient's symptoms were not improved. The blood of the infected patient C was collected in cooperation with the medical staff and the patient, and this was mixed with the reagent 1 of Preparation Example 1. Ceftriaxone, doxycycline and azithromycin were administered alone or in combination, and the SpO₂ concentration was measured after induction of the reaction for 1 hour.

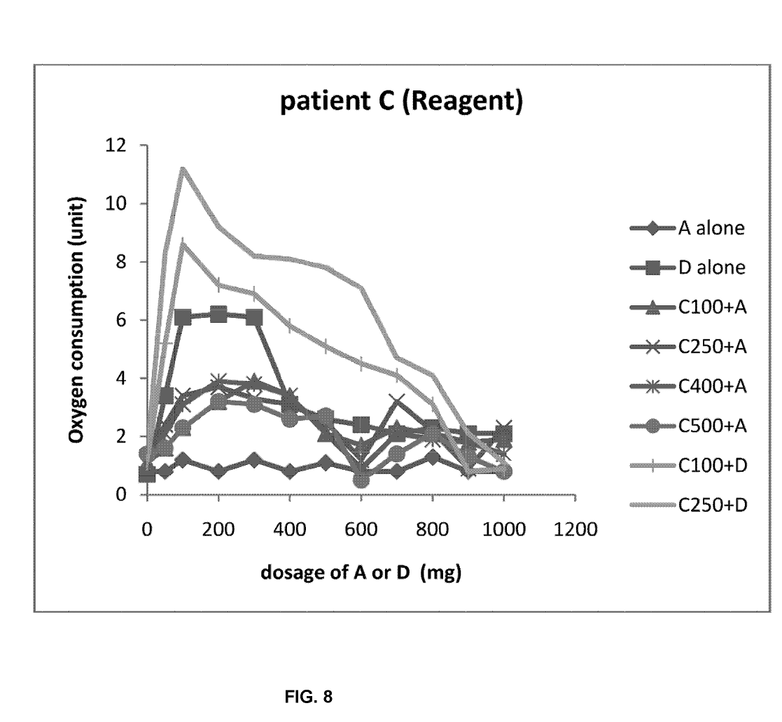

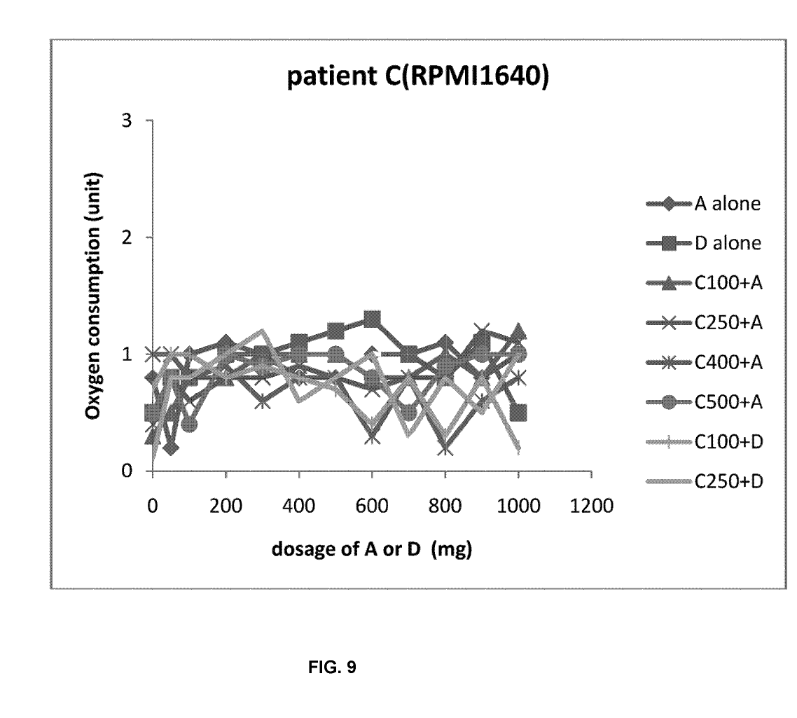

As a result, it was confirmed that, among the experimental groups using the first reagent, the consumption concentration of O₂ in the group treated with ceftriaxone 259 mg and doxycycline 100 mg was significantly higher (FIG. 8). On the other hand, no significant difference was observed in the O₂ consumption concentration depending on the drug in the control group (FIG. 9). Therefore, the medical staff treated patient C with doxycycline instead of azithromycin (in other words, the combined administration of ceftriaxone and doxycycline); patient C recovered after two weeks of treatment (A: azithromycin, C: ceftriaxone, D: doxycycline).

### EXPERIMENTAL EXAMPLE 4

### Testing of Patient Administered Anticancer Drugs

Patient D with chronic myelogenous leukemia was undergoing chemotherapy with imatinib (400 mg) and nilotinib (2×300 mg; that is, 300 mg twice per day), but the patient's symptoms were not improved.

The blood of patient D with chronic myelogenous leukemia was collected in cooperation with the medical staff and the patient, and this was mixed with the first reagent of Preparation Example 1. Imatinib, nilotinib, and dasatinib were administered alone or in a particular combination, and the SpO₂ concentration was measured after induction of the reaction for 1 hour.

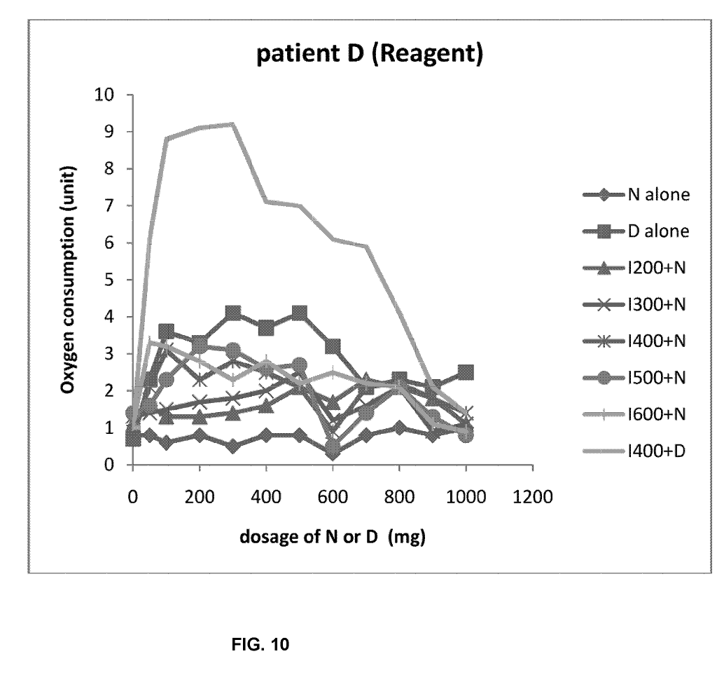

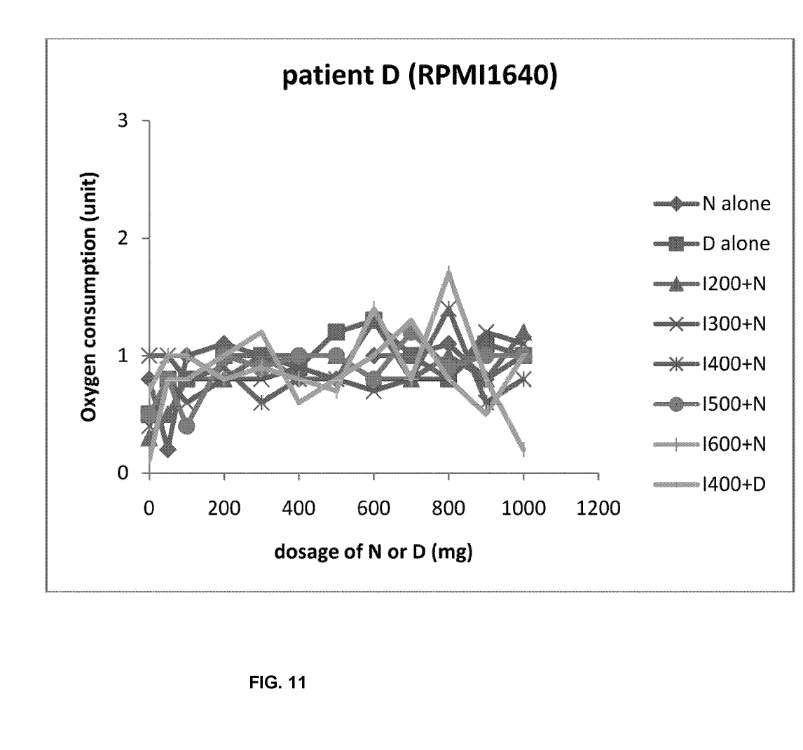

As a result, it was confirmed that, among the experimental groups using the first reagent, the consumption concentration of O₂ in the group treated with imatinib 400 mg and dasatinib 300 mg was significantly higher (FIG. 10). On the other hand, no significant difference was observed in the O₂ consumption concentration depending on the drug in the control group (FIG. 11). Therefore, the medical staff treated patient D with dasatinib instead of nilotinib (in other words, combined administration of imatinib and dasatinib); patient D experienced improved symptoms after two weeks of treatment (D: dasatinib, N: nilotinib, I: imatinib).

### EXPERIMENTAL EXAMPLE 5

### Testing of Patient Administered Anticancer Drugs

Patient E with chronic myelogenous leukemia was undergoing chemotherapy with imatinib (400 mg) and nilotinib (2×300 mg), but the patient's symptoms were not improved. The blood of patient E with chronic myelogenous leukemia was collected in cooperation with the medical staff and the patient, and this was mixed with the first reagent of Preparation Example 1. Imatinib, nilotinib, and dasatinib were administered alone or in a particular combination, and the SpO₂ concentration was measured after induction of the reaction for 1 hour.

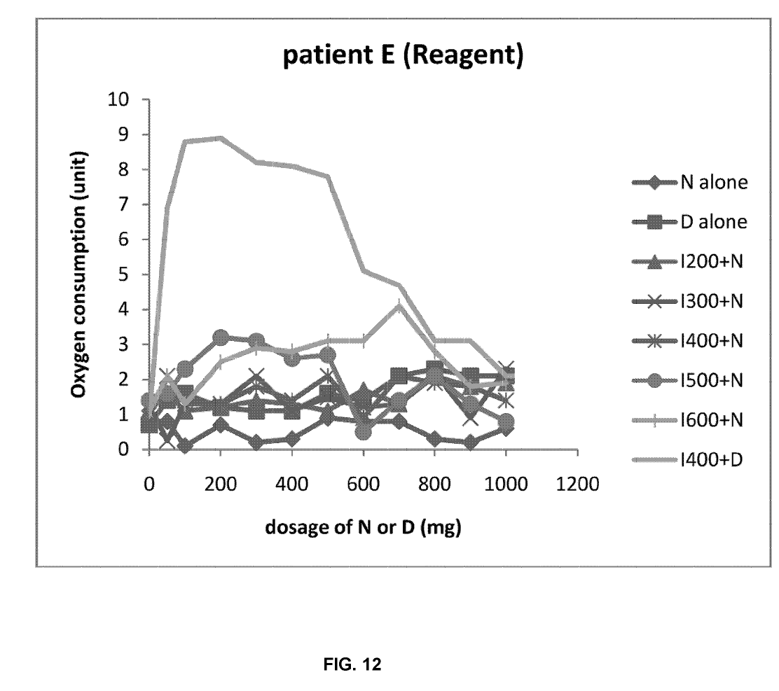

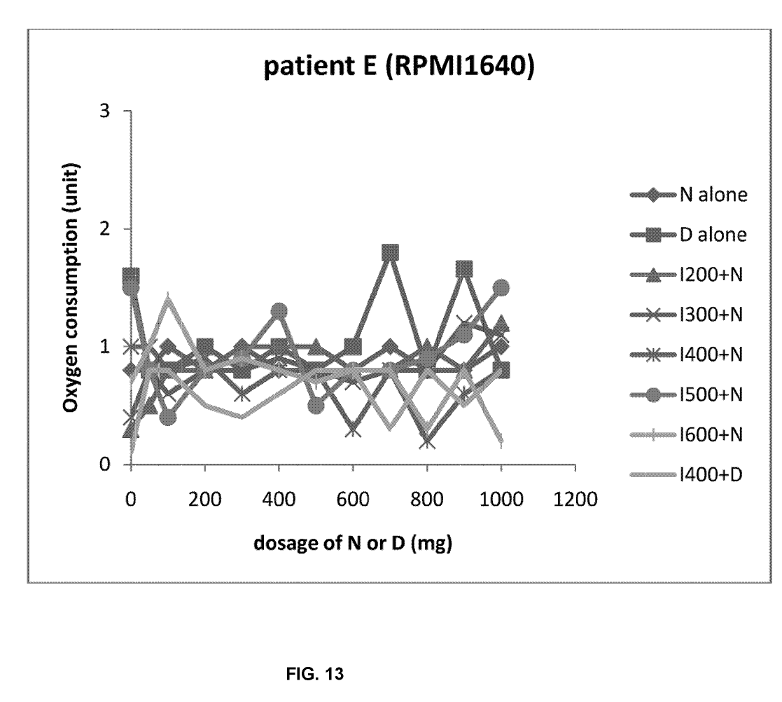

As a result, it was confirmed that, among the experimental groups using the first reagent, the consumption concentration of O₂ in the group treated with imatinib 400 mg and dasatinib 300 mg was significantly higher (FIG. 12). On the other hand, no significant difference was observed in the O₂ consumption concentration depending on the drug in the control group (FIG. 13). Therefore, the medical staff treated patient E with dasatinib instead of nilotinib (in other words, the combined administration of imatinib and dasatinib); patient E experienced improved symptoms after two weeks of treatment (D: dasatinib, N: nilotinib, I: imatinib).

### EXPERIMENTAL EXAMPLE 6

### Testing of Patient Administered Anticancer Drugs

Patient F with chronic myelogenous leukemia was undergoing chemotherapy with imatinib (400 mg) and nilotinib (2×300 mg), but the patient's symptoms were not improved. The blood of patient F with chronic myelogenous leukemia was collected in cooperation with the medical staff and the patient, and this was mixed with the first reagent of Preparation Example 1. Imatinib, nilotinib, and dasatinib were administered alone or in a particular combination, and the SpO₂ concentration was measured after induction of the reaction for 1 hour.

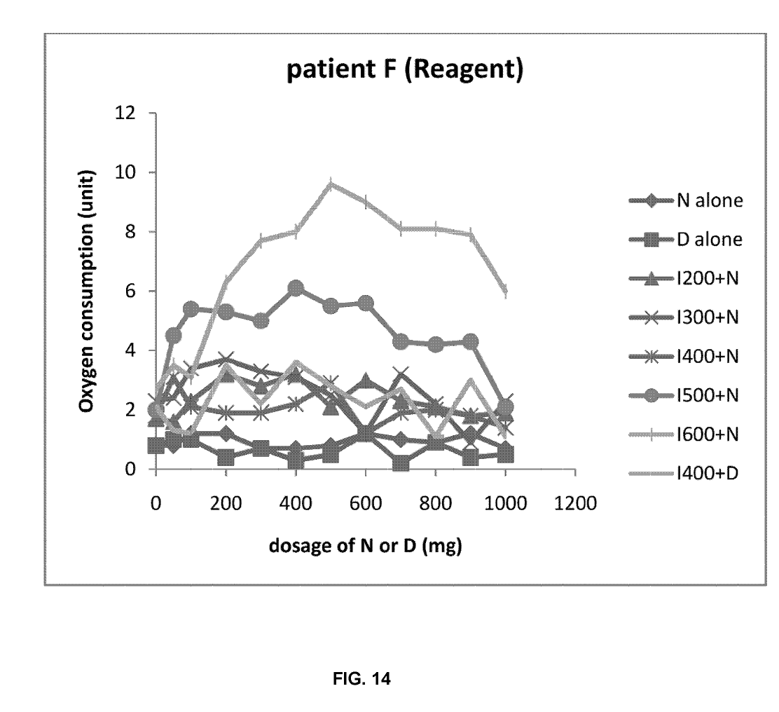

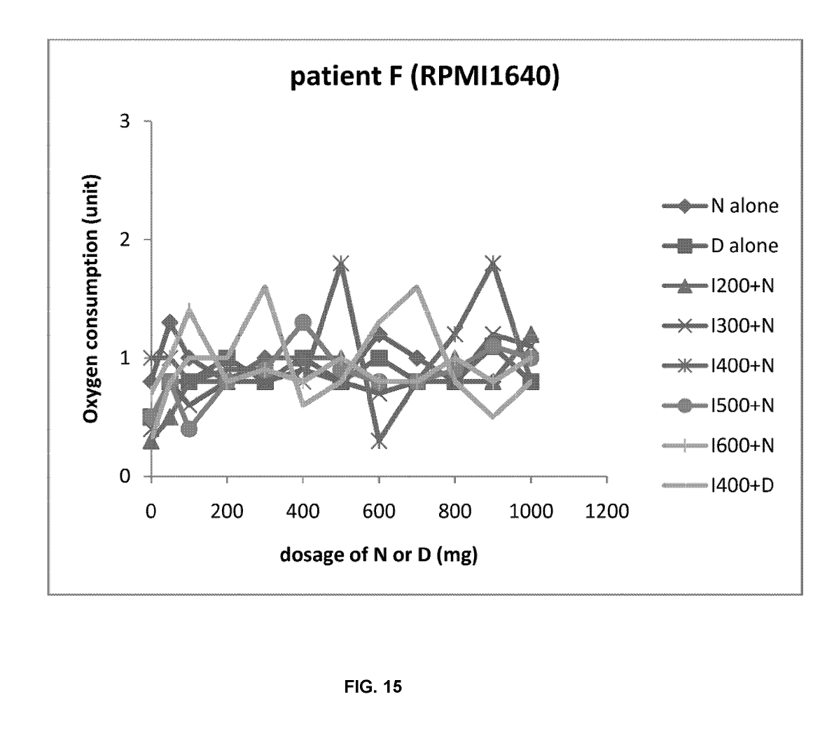

As a result, it was confirmed that, among the experimental groups using the first reagent, the consumption concentration of O₂ in the group treated with imatinib 600 mg and nilotinib 500 mg was significantly higher (FIG. 14). On the other hand, no significant difference was observed in the O₂ consumption concentration depending on the drug in the control group (FIG. 15).

Therefore, the medical staff increased the dose of imatinib as well as of nilotinib to patient F and administered them in combination; patient F experienced improved symptoms after two weeks of treatment (D: dasatinib, N: nilotinib, I: imatinib).

### EXPERIMENTAL EXAMPLE 7

### Testing of Patient Administered Anticancer Drugs

Patient G with chronic myelogenous leukemia was undergoing chemotherapy with imatinib (400 mg) and nilotinib (2×300 mg), but the patient's symptoms were not improved. The blood of patient G with chronic myelogenous leukemia was collected in cooperation with the medical staff and the patient, and this was mixed with the first reagent of Preparation Example 1. Imatinib, nilotinib, and dasatinib were administered alone or in a particular combination, and the SpO₂ concentration was measured after induction of the reaction for 1 hour.

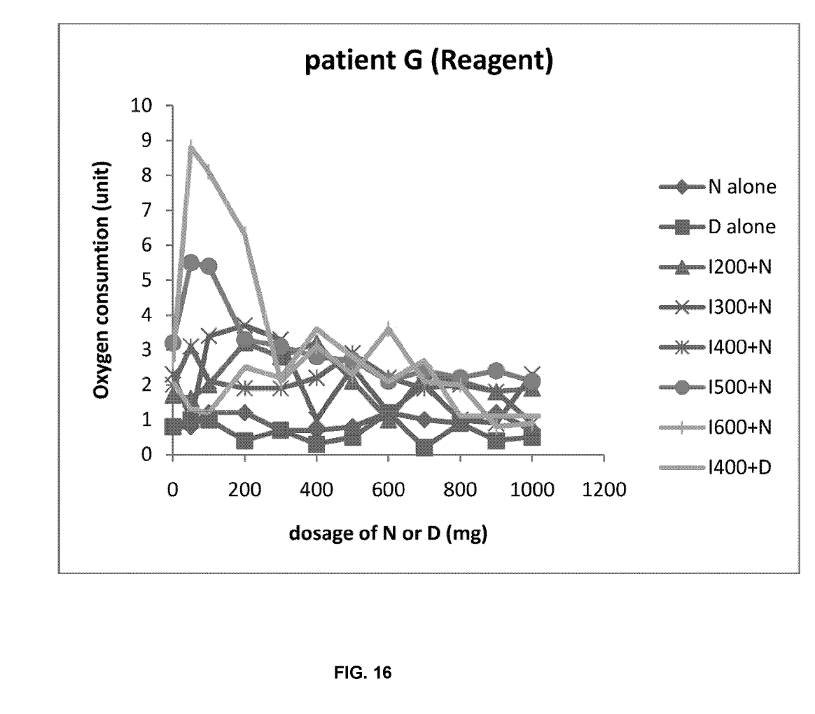

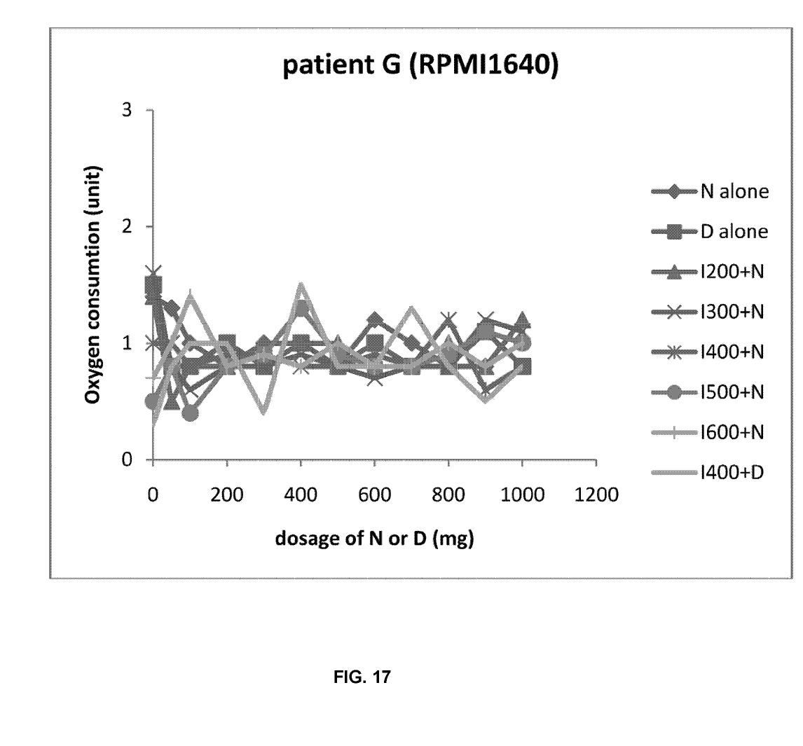

As a result, it was confirmed that, among the experimental groups using the first reagent, the consumption concentration of O₂ in the group treated with imatinib 600 mg and nilotinib 100 mg was significantly higher (FIG. 16). On the other hand, no significant difference was observed in the O₂ consumption concentration depending on the drug in the control group (FIG. 17). Therefore, the medical staff increased the dose of imatinib and decreased the dose of nilotinib to patient G and administered them in combination; patient G experienced improved symptoms after two weeks of treatment (D: dasatinib, N: nilotinib, I: imatinib).

### EXPERIMENTAL EXAMPLE 8

### Testing of Patient Administered Antibiotics and Anti-Inflammatory Drugs

The blood of the ARF patient H was collected and mixed with the first reagent of Preparation Example 1. Aspirin, corticosteroid and penicillin were administered alone or in a particular combination, and the SpO₂ concentration was measured after induction of the reaction for 1 hour.

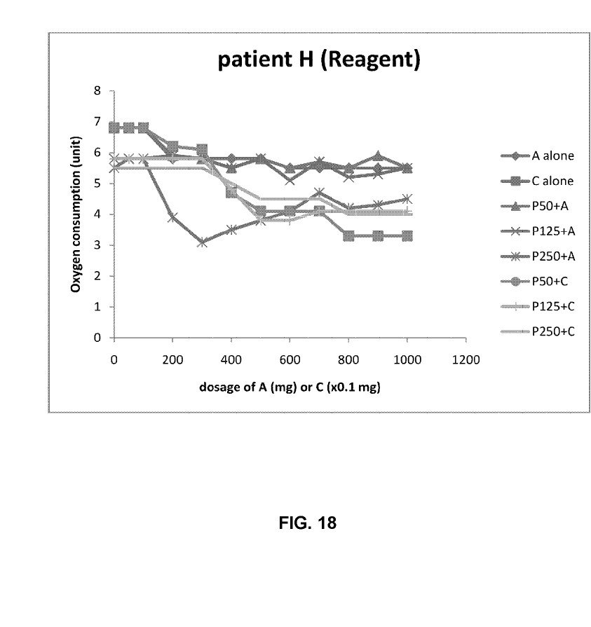

As a result, it was confirmed that the consumption concentration of O₂ in the group treated with aspirin 200 mg and penicillin 250 mg was significantly lower (FIG. 18); this was reflected in the prescription of the drug to the patient.

### EXPERIMENTAL EXAMPLE 9

### Testing of Patient Administered Antibiotics and Anti-Inflammatory Drugs

The blood of the ARF patient I was collected and mixed with the first reagent of Preparation Example 1. Aspirin, corticosteroid and penicillin were administered alone or in a particular combination, and the SpO₂ concentration was measured after induction of the reaction for 1 hour.

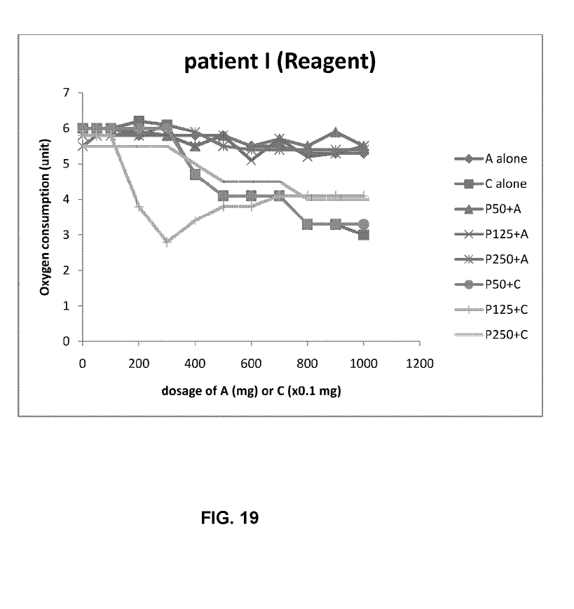

As a result, it was confirmed that the consumption concentration of O₂ in the group treated with aspirin 300 mg and penicillin 125 mg was significantly lower (FIG. 19); this was reflected in the prescription of the drug to the patient.

### EXPERIMENTAL EXAMPLE 10

### Testing of Patient Administered Antibiotics and Anti-Inflammatory Drugs

The blood of the ARF patient J was collected and mixed with the first reagent of Preparation Example 1. Aspirin, corticosteroid and penicillin were administered alone or in a particular combination, and the SpO₂ concentration was measured after induction of the reaction for 1 hour.

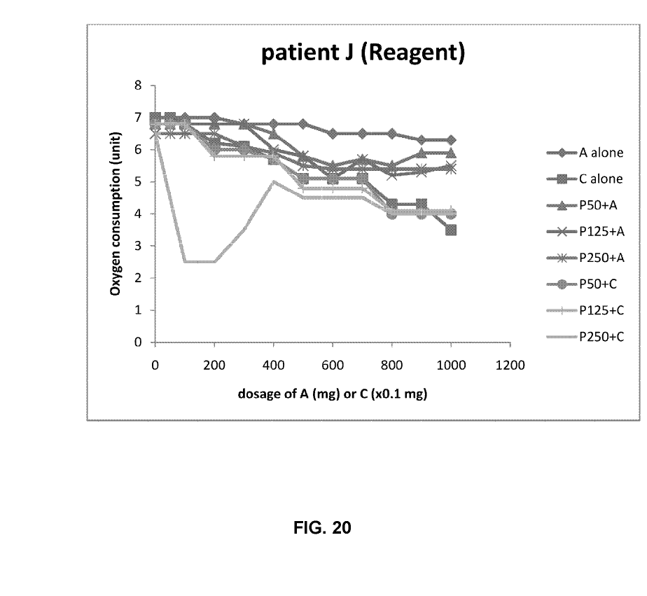

As a result, it was confirmed that the consumption concentration of O₂ in the group treated with corticosteroid 100 mg and penicillin 250 mg was significantly lower (FIG. 20); this was reflected in the prescription of the drug to the patient.

1.-10. (canceled)

11. A method for evaluating the immune response induction ability of a drug, comprising:
obtaining a mixture comprising:
a composition comprising a vitamin B group, vitamin D and phosphate-buffered saline (PBS);
blood of a subject; and
a drug to be prescribed for the subject; and
measuring consumption of oxygen in the mixture to determine the immune response induction ability.

12. The method of claim 11, wherein the composition further comprises at least one selected from the group consisting of aprotinin, fetuin, transferrin, calcium, selenium, thrombin inhibitor and pyruvate.

13. The method of claim 11, wherein the composition further comprises one or more selected from the group consisting of a mixture of vitamins A, C, E and K, heparin, deldeparin sodium, argatroban, bivalirudin, Lepirudin, serum albumin and immunoglobulin.

14. The method of claim 11, wherein the composition comprises vitamin B group, vitamin D and PBS in a volume ratio of 10:0.5 to 3:800 to 1300.

15. The method of claim 11, wherein the vitamin B group comprises two or more selected from the group consisting of vitamin B1, vitamin B2, vitamin B3, vitamin B6, and vitamin B12.

16. A method for evaluating the immune response induction ability of a drug, comprising:
obtaining a mixture comprising:
a composition comprising a vitamin B group, vitamin D and phosphate-buffered saline (PBS);
blood of a subject; and
a drug to be prescribed for the subject; and
measuring and quantifying oxygen consumption degree in the mixture.
